(19) United States
(12) Patent Application Publication
(10) Pub. No.: US 2023/0029570 A1
(43) Pub. Date: Feb. 2, 2023

(54) Bacillus Strain for Applications in Agriculture, Livestock Health and Environmental Protection

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/771,704

(22) PCT Filed: Apr. 13, 2021
(86) PCT No.: PCT/US2021/027041

§ 371 Date: Apr. 25, 2022

Related U.S. Application Data
(60) Provisional application No. 63/009,497, filed on Apr. 14, 2020.

(51) Int. Cl. C12N 1/20; C05F 11/08; A23K 10/18

(52) U.S. Cl. CPC C12N 1/205; C05F 11/08; A23K 10/18; C12R 2001/07

(57) ABSTRACT

A bacterial strain with enhanced biosurfactant-production capabilities is provided, as well as methods of its use in, for example, agriculture, livestock husbandry and environmental protection. In a specific embodiment, the present invention is directed to a bacterial strain that has novel properties for producing a mixture of lipopeptides that is unique to its genus and species. Specifically, the bacterium is a novel strain of Bacillus amyloliquefaciens.

### CROSS-REFERENCE TO RELATED APPLICATION

This application claims priority to U.S. Provisional Patent Application No. 63/009,497 filed Apr. 14, 2020, which is incorporated herein by reference in its entirety.

### BACKGROUND OF THE INVENTION

Bacillus amyloliquefaciens is a species of aerobic bacteria discovered in soil in 1943. The name “amyloliquefaciens” comes from the bacterium's production of a “liquefying” alpha-amylase enzyme useful for, e.g., starch hydrolysis. In addition to amylase, B. amyloliquefaciens can produce enzymes including proteases, cellulases, lipases, mannanases, pectate lyases, and peroxidases/oxidases. Furthermore, B. amyloliquefaciens is also a known producer of lipopeptide biosurfactants, as well as other useful bioactive metabolites.

B. amyloliquefaciens is a Gram-positive, motile, rod-shaped bacterium that often forms chains. The optimal temperature for growth is 30 to 40° C., with no growth below 15° C. or above 50° C. The organism was previously described and distinguished from B. subtilis in Priest et al., Bacillus amyloliquefaciens sp. nov., nom. rev., Int'l J System Bacteriol, 37, 69-71 (1987) (incorporated by reference herein in its entirety). The organism has also been characterized as a low G+C organism, it has fewer guanine and cytosine bases than adenine and thymine bases in its DNA, compared to other bacteria.

The growth by-products of B. amyloliquefaciens, as well as the microbe itself, have the potential for a wide variety of uses in industry. Nonetheless, there is a continuous need to develop bacterial strains that exhibit improved properties for use in environmentally sustainable, non-toxic and biodegradable methods and products. Notably, industry applications such as agriculture, livestock husbandry, greenhouse gas reduction, detergents and cleaning supplies, and countless others, would benefit from novel bacterial strains that have enhanced properties.

### BRIEF SUMMARY OF THE INVENTION

The subject invention provides novel advantageous microbes, as well as by-products of their growth, such as biosurfactants. The subject invention also provides advantageous methods of using these novel microbes and their by-products in a variety of applications including, for example, promoting plant health and productivity; enhancing health of livestock and other animals; reducing greenhouse gas emissions from, for example, agriculture and livestock production; cleaning and/or disinfecting household materials and surfaces; and many others.

In some embodiments, the present invention provides a novel Bacillus amyloliquefaciens strain and by-products thereof. These by-products can include, for example, enzymes, biosurfactants, and other useful metabolites.

In preferred embodiments, the novel Bacillus amyloliquefaciens strain, referred to as “B. amyloliquefaciens var. locus,” “B. amyloliquefaciens subsp. locus” and/or “B. amy,” has unique characteristics compared to reference strains.

In some embodiments, B. amy is capable of thriving under saline conditions and can grow at temperatures of 55 ° C. and higher.

In some embodiments, B. amy is capable of producing a mixture of lipopeptide biosurfactants that is unique compared to natural B. amyloliquefaciens species, as well as to the Bacillus genus. Specifically, and advantageously, B. amy produces a unique mixture of surfactin, lichenysin, fengycin and iturin A.

In some embodiments, B. amy is a “biosurfactant over-producing” strain. For example, the strain may produce at least 0.1-10 g/L, e.g., 0.5-1 g/L total of one or more biosurfactants, or at least 10%, 25%, 50%, 100%, 2-fold, 5-fold, 7.5 fold, 10-fold, 12-fold, 15-fold or more the total amount of biosurfactant(s) compared to the total amount of biosurfactant(s) produced by reference Bacillus amyloliquefaciens strains, such as, e.g., B. amyloliquefaciens IT-45.

In some embodiments, B. amy is capable of producing glycolipid biosurfactants, phytase, organic acids, nitrogen fixation enzymes and/or growth hormones.

In certain embodiments, the subject invention provides materials and methods for enhancing plant growth, health and productivity by applying a soil treatment composition comprising B. amy to the plant and/or the plant's surrounding environment. In preferred embodiments, the method comprises applying B. amy in combination with one or more additional microbes, such as, for example, Trichoderma harzianum. Particularly, by enhancing the health and growth of plant roots, the synergistic combination of B. amy and T. harzianum is especially effective for boosting the productivity of a wide variety of crops, including, for example, citrus, potatoes, corn, lettuce, hemp, turf, strawberries, tobacco, melons, and almonds.

Advantageously, in some embodiments, the B. amy soil treatment composition can also improve one or more properties of the rhizosphere, such as, for example, salinity, pollutant content, moisture retention, drainage, and nutrient dispersal; and/or promote the formation of carbon sinks in soil by enhancing the sequestration of carbon in the soil, in above- and below-ground plant biomass, and in soil microbial biomass.

In certain embodiments, the subject invention provides materials and methods for enhancing the health of livestock and other animals by applying a B. amy composition to the digestive system of the livestock or other animal. For example, B. amy can function as a probiotic, to enhance body weight gain, to promote feed intake and conversion, and to increase growth hormone levels. Additionally, B. amy can promote the growth of other beneficial microbes (e.g., fatty acid producers) while decreasing the amount of potential pathogenic and/or methanogenic microbes in an animal's gut.

Advantageously, when administered to the digestive system of an animal, B. amy can also be useful for the control of methanogens and/or protozoa present in the digestive system and/or waste products of the animal. Thus, the B. amy composition and methods can also be used for reducing the production of enteric greenhouse gases (e.g., methane and carbon dioxide) and/or greenhouse gas precursors (e.g., organic nitrogen).

In one embodiment, the subject invention provides methods of producing a microbial growth by-product by cultivating B. amy under conditions appropriate for growth and production of the growth by-product(s); and, optionally, extracting, concentrating and/or purifying the growth by-product(s). The growth by-product can be, for example, one or more biosurfactants, enzymes, solvents, biopolymers, proteins, amino acids, gases, and/or other metabolites.

In specific embodiments, the growth by-product is a lipopeptide biosurfactant, or a mixture of lipopeptide biosurfactants. In one embodiment, the mixture of lipopeptides comprises surfactin, fengycin, lichenysin and/or iturin. This lipopeptide mixture can be useful in a variety of applications, including, for example, as part of an environmentally-friendly disinfectant cleaning composition.

In one embodiment, the method of producing a microbial growth by-product comprises cultivating B. amy in the presence of Myxococcus xanthus, wherein such co-cultivation results in enhanced production of the growth by-product compared to when a strain of B. amyloliquefaciens is cultivated individually.

In some embodiments, the microbes and microbe-based products of the subject invention can be useful in a variety of applications as “green,” or environmentally-friendly, alternatives to, for example, chemical products. These can include, but are not limited to, agriculture, livestock domestic pets, rearing, forestry, turf and pasture management, aquaculture, mining, waste disposal and treatment, environmental remediation, human health, cosmetics, oil and gas recovery, and others listed herein.

### DETAILED DESCRIPTION

In some embodiments, the present invention provides a novel strain of Bacillus amyloliquefaciens and growth by-products thereof. These growth by-products can include, for example, biosurfactants, enzymes, and other metabolites.

In preferred embodiments, the strain, B. amy, is characterized by its ability to produce a unique lipopeptide mixture comprising surfactin, lichenysin, fengycin and/or iturin A, which is a characteristic that is not present in natural Bacillus amyloliquefaciens strains. In further preferred embodiments, the strain is characterized by enhanced biosurfactant production compared to reference Bacillus amyloliquefaciens strains. In yet further preferred embodiments, the strain is characterized by the ability to produce one or more of glycolipid biosurfactants, phytase, organic acids, nitrogen fixation enzymes and growth hormones.

In some embodiments, B. amy can survive and grow under saline conditions and at temperatures of 55° C. or greater.

The subject invention further provides methods of cultivating B. amy and its growth by-products, as well as methods of their use in, for example, agriculture, livestock husbandry, forestry, turf and pasture management, aquaculture, mining, waste disposal and treatment, environmental remediation, human health, cosmetics, and oil and gas recovery.

### Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The cells may be in a vegetative state or in spore form, or a mixture of both. The cells may be planktonic or in a biofilm form, or a mixture of both. The by-products of growth may be, for example, metabolites, cell membrane components, proteins, and/or other cellular components. The cells may be intact or lysed. In some embodiments, the cells are present, with broth in which they were grown, in the microbe-based composition. The cells may be present at, for example, a concentration of at least 1×10⁴, 1×10⁵, 1×10⁶, 1×10′, 1×10⁸, 1×10⁹, 1×10¹⁰, 1×10¹¹ or 1×10¹² or more cells per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, buffers, appropriate carriers, such as water, added nutrients to support further microbial growth, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

As used here in, a “biologically pure culture” is one that has been isolated from biologically active materials, including any materials with which it may have been associated with in nature. In a preferred embodiment, the culture has been isolated from all other living cells. In further preferred embodiments, the biologically pure culture has advantageous characteristics compared to a culture of the same microbial species that may exist in nature. The advantageous characteristics can be, for example, enhanced production of one or more desirable growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “applying” a composition or product refers to applying it to a target or site such that the composition or product can have an effect on that target or site. The effect can be due to, for example, microbial growth and/or the action of a biosurfactant or other growth by-product. In certain embodiments, B. amy can be applied to a target or site in live form, inactive form, dormant form, vegetative form, or spore form, or a mixture thereof. In certain embodiments, B. amy can be applied to a target or site in combination with one or more other microorganisms, such as, for example, Trichoderma harzianum, Trichoderma viride, Azotobacter vinelandii, Frateuria aurantia, Myxococcus xanthus, Pseudomonas chlororaphis, Wickerhamomyces anomalus, Starmerella bombicola, Saccharomyces cerevisiae, Saccharomyces boulardii, Pichia occidentalis, Pichia kudriavzevii, Meyerozyma guilliermondii, Pleurotus ostreatus, Lentinula edodes, Monascus purpureus, Acremonium chrysogenum, Bacillus subtilis and/or Bacillus licheniformis.

As used herein, an “alteration” in expression means a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

As used here, a “host cell” refers to a cell, such as a microorganism cell, that is to be, or has been, transformed with exogenous (non-host) DNA, using the methods and compositions of the invention.

As used herein, “transformation” refers to a permanent or transient genetic change, preferably a permanent genetic change, induced in a cell following incorporation of one or more non-host nucleic acid sequences. Transformation (including transduction or transfection), can be achieved by any one of a number of means including electroporation, conjugation, microinjection, biolistics (or particle bombardment-mediated delivery), or agrobacterium-mediated transformation.

As used herein, “vector” generally refers to a polynucleotide that can be propagated and/or transferred between organisms, cells, or cellular components. Vectors include viruses, bacteriophage, pro-viruses, plasmids, phagemids, transposons, and artificial chromosomes, that are able to replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium.

As used herein, “promoter” refers to a minimal nucleic acid sequence sufficient to direct transcription of a nucleic acid sequence to which it is operably linked. The term “promoter” is also meant to encompass those promoter elements sufficient for promoter-dependent gene expression controllable for cell-type specific expression or inducible by external signals or agents; such elements may be located in the 5′ or 3′ regions of the naturally-occurring gene.

“Engineering” or “modifying” a microorganism can include the introduction of a genetic material into a host or parental microorganism, and/or the disruption, deletion, or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new or greater quantities of an intracellular metabolite, improve the flux of a metabolite down a desired pathway, and/or reduce the production of undesirable by-products).

Microorganisms provided herein are able to produce certain metabolites in quantities and/or combinations not available in reference organisms of the same species. A “metabolite” refers to any substance produced by metabolism (e.g., a growth by-product) or a substance necessary for taking part in a particular metabolic process. A metabolite can be an organic compound that is a starting material, an intermediate in, or an end product of metabolism.

As used herein, a “fragment” of a polypeptide or nucleic acid molecule means a portion thereof. This portion contains, preferably, at least 10%, 20%, 30%, 40%, 50%, 60%, 70%, 80%, 90%, 95%, or 99% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids, or more.

As used herein, a “gene” is a locus (or region) of DNA that encodes a functional RNA or protein product.

As used herein, “modulate” means to alter (increase or decrease). Such alterations are detected by standard art known methods such as those described herein.

Nucleic acids include but are not limited to: deoxyribonucleic acid (DNA), ribonucleic acid (RNA), double-stranded DNA (dsDNA), single-stranded DNA (ssDNA), messenger RNA (mRNA), ribosomal RNA (rRNA), transfer RNA (tRNA), micro RNA (miRNA), and small interfering RNA (siRNA).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, a “reference” condition or material is a standard or control condition or material. For example, a “reference strain” is a wild-type strain of a microorganism, or a strain of a microorganism that is acquired from a culture type collection. In some embodiments, B. amyloliquefaciens IT-45 is used as a reference strain according to the subject invention.

As another example, as used herein, a “reference sequence” is a defined sequence used as a basis for sequence comparison or a gene expression comparison. A reference sequence may be a subset of, or the entirety of, a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

As used herein, a polypeptide or nucleic acid molecule that is “substantially identical” to a reference exhibits at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% or more identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wisc. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, “obtaining” as in, for example, “obtaining an agent” includes synthesizing, purchasing, or otherwise acquiring the agent.

As used herein, “salt-tolerant” means capable of growing in a sodium chloride concentration of at least 10%, 12%, 15%, or greater. In a specific embodiment, “salt-tolerant” refers to the ability to grow in 100 to 150 g/L or more of NaCl.

As used herein, a “surfactant” is a compound that lowers the surface tension (or interfacial tension) between two interfaces (e.g., between a liquid and a liquid, or a liquid and a solid). Surfactants act as, for example, detergents, wetting agents, emulsifiers, foaming agents, and dispersants. A “biosurfactant” is a surface-active substance produced by a living cell.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Bacillus amyloliquefaciens var. locus (“B. amy”)

The Bacillus microorganisms exemplified herein have been characterized and classified as Bacillus amyloliquefaciens. B. amy is a genetically-modified strain, which was confirmed by whole genome sequencing and de novo assembly.

A culture of the B. amyloliquefaciens “B. amy” microbe has been deposited with the Agricultural Research Service Northern Regional Research Laboratory (NRRL), 1400 Independence Ave., S.W., Washington, D.C., 20250, USA. The deposit has been assigned accession number NRRL B-67928 by the depository and was deposited on Feb. 26, 2020.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122.

The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

The B. amy strain developed according to the current invention produces a mixture of lipopeptide biosurfactants that is unique when compared with biosurfactant production capabilities of reference strains of B. amyloliquefaciens, as well as all Bacillus spp. This lipopeptide mixture comprises surfactin, lichenysin, fengycin and iturin A.

In some embodiments, B. amy produces greater total amounts of biosurfactants compared to reference strains of Bacillus amyloliquefaciens. In some embodiments, the biosurfactant-producing abilities of the microbe (i.e., the type(s) and/or amount(s) of biosurfactants produced) can be controlled by altering the nutrient composition of the medium. The strain can be grown using solid state and submerged fermentation methods to produce high cell counts and high metabolite content.

In some embodiments, B. amy survives and grows under high saline conditions and at temperatures of 55° C. or higher. The strain is also capable of growing under anaerobic conditions. The B. amy strain can also be used for producing enzymes that degrade or metabolize starches.

In some embodiments, B. amy is capable of producing glycolipid biosurfactants, phytase, organic acids, nitrogen fixation enzymes and/or growth hormones.

The microbe can be grown in planktonic form or as biofilm. In the case of biofilm, the vessel may have within it a substrate upon which the microbe can be grown in a biofilm state. The microbe may be induced into a biofilm state using techniques known in the art. The system may also have, for example, the capacity to apply stimuli (such as shear stress) that encourages and/or improves the biofilm growth characteristics.

B. amy can be readily identified using methods known in the art, including, for example, PCR primer pairs and 16s sequencing.

### Use of the Subject Microbes for Production of Growth By-Products

In one embodiment, the subject invention provides methods of producing a microbial growth by-product by cultivating B. amy under conditions appropriate for growth and production of the growth by-product; and, optionally, extracting, concentrating and/or purifying the growth by-product. The growth by-product can be, for example, one or more biosurfactants, enzymes, solvents, biopolymers, proteins, amino acids, gases, and/or other metabolites.

In a specific embodiment, the B. amy microbes of the subject invention can be used to produce one or more biosurfactants.

Biosurfactants are a structurally diverse group of surface-active molecules produced by microorganisms. Biosurfactants are amphiphilic molecules consisting of both hydrophobic (e.g., a fatty acid) and hydrophilic domains (e.g., a sugar). These molecules are unique in that they are produced via microbial fermentation but have those properties possessed by chemical surfactants in addition to other attributes not possessed by their synthetic analogs. Due to their amphiphilic nature, biosurfactants can partition at the interfaces between different fluid phases such as oil/water or water/air interfaces.

In some embodiments, the biosurfactants produced by B. amy are advantageous due to their reduced micelle size compared with, for example, the size of synthetic surface-active compounds. A small micelle size can be effective for penetrating cell membranes and intercellular spaces (e.g., the blood brain barrier), biofilms, and other nanoscale sized spaces and pores to benefit the health of humans, plants and animals in a variety of ways.

In certain embodiments, the size of a biosurfactant molecule and/or a biosurfactant micelle according to the subject invention is less than 10 nm, preferably less than 8 nm, more preferably less than 5 nm. In a specific embodiment, the size is from 0.8 nm to 1.5 nm, or about 1.0 to 1.2 nm.

In some embodiments, penetration of nano-sized biosurfactants and/or biosurfactant micelles into cells results in a reduction in surface/interfacial tension on both the inside and outside of cells. Advantageously, in some embodiments, this facilitates the transport of beneficial compounds into cells, such as, e.g., water, drugs and nutrients, and also facilitates the transport of detrimental compounds out of cells, such as, e.g., waste products, toxins, and DNA-damaging free radicals. Thus, the biosurfactants can contribute to enhanced cell health and enhanced overall health for humans, plants and animals.

In some embodiments, the size of biosurfactants and/or biosurfactant micelles facilitates penetration thereof into biofilms matrices, thereby enhancing the disruption of biofilms on surfaces inside and outside of human and animal bodies and plants.

The biosurfactants can be produced using solid state fermentation, submerged fermentation and/or combinations thereof. Biosurfactants according to the subject invention can include, for example, glycolipids, lipopeptides, flavolipids, phospholipids, fatty acid esters, and high-molecular-weight biopolymers such as lipoproteins, lipopolysaccharide-protein complexes, and/or polysaccharide-protein-fatty acid complexes.

In one embodiment, the biosurfactant is a lipopeptide, such as, for example, surfactin, iturin, fengycin, arthrofactin, amphisin, lichenysin, paenibacterin, polymyxin and/or battacin, plipastatin, kurstakins, bacillomycin, mycosubtilin, glomosporin, syringomycin and/or viscosin.

In some embodiments, the microorganisms can also produce one or more additional types of biosurfactants, such as glycolipids (e.g., rhamnolipids (RLP), sophorolipids (SLP), trehalose lipids, cellobiose lipids and/or mannosylerythritol lipids (MEL)), fatty acid esters (e.g., oleic fatty acid esters), saponins, cardiolipins, pullulan, emulsan, lipomanan, alasan, and/or liposan.

In one embodiment, the method of producing a microbial growth by-product comprises cultivating B. amy in the presence of Myxococcus xanthus, wherein such co-cultivation results in enhanced production of the growth by-product compared to when a strain of B. amylohquefaciens is cultivated individually. In certain embodiments, the growth by-product is a biosurfactant, including glycolipids, such as, for example, MEL, and/or lipopeptides, such as, for example, surfactin, iturin, lichenysin and/or fengycin.

In some embodiments, B. amy, cultivated on its own or with another microbe, can produce a mixture of lipopeptide biosurfactants comprising surfactin, fengycin, lichenysin and iturin A. In some embodiments, the majority (e.g., at least 50% of the lipopeptide mixture comprises surfactin). This lipopeptide mixture can be useful in a variety of applications, including, for example, as part of an environmentally-friendly disinfectant cleaning composition.

The biosurfactants produced by B. amy can be useful for a variety of industries, such as, for example, agriculture, livestock husbandry, cleaning and disinfecting products, greenhouse gas reduction, environmental remediation, human health and pharmaceuticals, food production and processing, cosmetics, oil and gas recovery, waste treatment, and countless others.

In one exemplary embodiment, B. amy and/or the biosurfactants it produces can be used to improve the health and productivity of plants undergoing water stress.

Biosurfactants decrease the tendency of water to pool, they improve the adherence or wettability of surfaces, which results in more thorough hydration of the full rhizosphere, and they reduce the volume of water that might otherwise escape below the root zone via micro-channels formed by drip and micro-irrigation systems. This ‘wettability’ also promotes improved root system health as there are fewer zones of desiccation (or extreme dryness) inhibiting proper root growth and better availability of applied nutrients as chemical and micro-nutrients are more thoroughly made available and distributed.

The more uniform distribution of water in the crop rhizosphere made possible by enhanced wettability also prevents water from accumulating or becoming trapped above optimal penetration levels thereby mitigating anaerobic conditions that inhibit the free exchange of oxygen and carbon. A more porous crop rhizosphere is established and roots will have greater resistance to soil borne disease. The combination of a properly hydrated and aerated rhizosphere also increases the susceptibility of soil pests and pathogens (such as nematodes and soil borne fungi and their spores) to chemical pesticides and biopesticides. Thus, the biosurfactants can be used for a wide range of useful applications include disease and pest control.

In another exemplary embodiment, B. amy and/or the biosurfactants it produces can be used to directly control a pest due to their antibacterial, antifungal, antinematodal and antiviral properties. In one embodiment, the pest is a pathogen that infects plants, animals and/or humans.

In another exemplary embodiment, B. amy and/or the biosurfactants it produces can be used to enhance the recovery of oil from an oil well by, for example, stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; control of corrosion-causing bacteria (e.g., SRB); reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and enhancement of fracturing fluids.

In yet another exemplary embodiment, B. amy and/or the biosurfactants it produces can be used as an organic food preservative to increase the consumable life of produce and processed foods.

In yet another exemplary embodiment, B. amy and/or the biosurfactants it produces can be used in a non-toxic disinfectant cleaning composition to control bacteria such as, for example, E. coli and Staph aureus present on household surfaces.

In addition to biosurfactants, the growth by-product can include other metabolites, for example, enzymes, enzyme inhibitors, biopolymers, acids, solvents, gases, proteins, peptides, amino acids, alcohols, pigments, pheromones, hormones, lipids, ectotoxins, endotoxins, exotoxins, carbohydrates, antibiotics, anti-fungals, anti-virals and/or other bioactive compounds.

Enzymes according to the subject invention can include, for example, oxidoreductases, transferases, hydrolases, lyases, isomerases and/or ligases. Specific types and/or subclasses of enzymes according to the subject invention can also include, but are not limited to, nitrogenases, proteases, amylases, glycosidases, cellulases, glucosidases, glucanases, galactosidases, moannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases (e.g., alkane hydroxylase), esterases, lipases, ligninases, mannanases, oxidases, laccases, tyrosinases, cytochrome P450 enzymes, peroxidases (e.g., chloroperoxidase and other haloperoxidases), and lactases.

In certain embodiments, the by-products include antibiotic compounds, such as, for example, aminoglycosides, amylocyclicin, bacitracin, bacillaene, bacilysin, bacilysocin, corallopyronin A, difficidin, etnangien gramicidin, β-lactams, licheniformin, macrolactinsublancin, oxydifficidin, plantazolicin, ripostatin, spectinomycin, subtilin, tyrocidine, and/or zwittermicin A. In some embodiments, an antibiotic can also be a type of biosurfactant.

In certain embodiments, the growth by-products include anti-fungal compounds, such as, for example, fengycin, surfactin, haliangicin, mycobacillin, mycosubtilin, and/or bacillomycin. In some embodiments, an anti-fungal can also be a type of biosurfactant.

In certain embodiments, the growth by-products include other bioactive compounds, such as, for example, butanol, ethanol, acetate, ethyl acetate, lactate, acetoin, benzoic acid, 2,3-butanediol, beta-glucan, indole-3-acetic acid (IAA), lovastatin, aurachin, kanosamine, reseoflavin, terpentecin, pentalenolactone, thuringiensin (β-exotoxin), polyketides (PKs), terpenes, terpenoids, phenyl-propanoids, alkaloids, siderophores, as well as ribosomally and non-ribosomally synthesized peptides, to name a few.

The microbial growth by-products produced by the subject strain may be retained in the microorganisms or secreted into the medium in which the strain is cultivated. In some embodiments, the microbial growth by-product may further be extracted, concentrated and/or purified.

Advantageously, in accordance with the subject invention, a microbe-based product produced according to the subject invention may comprise the microbes in broth (or other medium components) in which the microbes were grown, as well as the microbial growth by-products and any residual nutrients. The product may be, for example, at least, by weight, 1%, 5%, 10%, 25%, 50%, 75%, or 100% broth (or other medium). The amount of biomass in the product, by weight, may be, for example, 0% to 50%, 5% to 60%, 10% to 70%, 20% to 80%, 30% to 90%, or 0% to 100%. The amount of growth by-product in the product, by weight, may be, for example, 0% to 50%, 5% to 60%, 10% to 70%, 20% to 80%, 30% to 90%, at least 91%, at least 92%, at least 93%, at least 94%, at least 95%, at least 96%, at least 97%, at least 99% or about 100%.

### Use of the Subject Microbe Strain as a Soil Treatment

In one embodiment, B. amy can be used as a microbial soil treatment. When applied to, for example, seed, plant, or soil of row crops, forestry operations, managed pastures, horticulture crops, managed turf, or other plant environments, the inoculant becomes an integral part of the property of the host soil or host medium and promotes the healthy growth of indigenous, beneficial microorganisms that benefit that soil or medium or plants and animals that are grown, fed or otherwise exposed to these soils and media.

In certain embodiments, the subject invention provides materials and methods for enhancing plant growth, health and productivity by applying B. amy to the plant and/or the plant's surrounding environment.

As used herein, “enhancing” means improving or increasing. For example, enhanced plant health means improving the plant's ability grow and thrive, which includes increased seed germination and/or emergence, improved ability to ward off pests and/or diseases, and improved ability to survive environmental stressors, such as droughts and/or overwatering. Enhanced plant growth and/or enhanced plant biomass means increasing the size and/or mass of a plant both above and below the ground (e.g., increased canopy/foliar volume, height, trunk caliper, branch length, shoot length, protein content, root size/density and/or overall growth index), and/or improving the ability of the plant to reach a desired size and/or mass. Enhanced yields mean improving the end products produced by the plants in a crop, for example, by increasing the number, amount and/or size of fruits, leaves, roots, extracts, and/or tubers per plant, and/or improving the quality of the fruits, leaves, roots and/or tubers (e.g., improving taste, texture, brix, chlorophyll content, cannabinoid content and/or color).

The “surrounding environment” of a plant means the environment sufficiently close to the plant so that the composition may contact the plant such that the desired result (e.g., killing a pest, increasing yield, preventing damage to the plant, regulating genes and/or hormones, etc.) is achieved. This may typically be within, for example, 50, 10, 5, 3, 2, or 1 feet or less, of the desired target.

In preferred embodiments, the method comprises applying B. amy in combination with one or more additional microorganisms to the plant's roots and/or to the soil in which the plant is, or will be, planted. The B. amy can also be applied in combination with micronutrients and/or prebiotic starter materials including, for example, humic acid, kelp extract, humate and/or fulvic acid.

In a specific embodiment, the one or more additional microorganisms is Trichoderma harzianum. By enhancing the health and growth of plant roots, the synergistic combination of B. amy and T. harzianum is especially effective for boosting the productivity of a wide variety of crops, including, for example, citrus, potatoes, corn, lettuce, hemp, turf, strawberries, tobacco, melons, and almonds.

In certain embodiments, the one or more additional microorganisms are yeasts and/or fungi, which include, for example, Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Meyerozyma spp. (e.g., M. guilliermondii), Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus), Williopsis (e.g., W. mrakii), Zygosaccharomyces (e.g., Z. bailii), mycorrhizal fungi and others. As used herein, “mycorrhizal fungi” includes any species of fungus that forms a non-parasitic mycorrhizal relationship with a plant's roots. The fungi can be ectomycorrhizal fungi and/or endomycorrhizal fungi, including subtypes thereof (e.g., arbuscular, ericoid, and orchid mycorrhizae).

Non-limiting examples of mycorrhizal fungi according to the subject invention include species belong to Glomeromycota, Basidiomycota, Ascomycota, Zygomycota, Helotiales, and Hymenochaetales, as well as Acaulospora spp. (e.g., A. alpina, A. brasiliensis, A. foveata), Amanita spp. (e.g., A. muscaria, A. phalloides), Amphinema spp. (e.g., A. byssoides, A. diadema, A. rugosum), Astraeus spp. (e.g., A. hygrometricum), Byssocorticium spp. (e.g., B. atrovirens), Byssoporia terrestris (e.g., B. terrestris sartoryi, B. terrestris lilacinorosea, B. terrestris aurantiaca, B. terrestris sublutea, B. terrestris parksii), Cairneyella spp. (e.g., C. variabilis), Cantherellus spp. (e.g., C. cibarius, C. minor, C. cinnabarinus, C. friesii), Cenococcum spp. (e.g., C. geophilum), Ceratobasidium spp. (e.g., C. cornigerum), Cortinarius spp. (e.g., C. austrovenetus, C. caperatus, C. violaceus), Endogone spp. (e.g., E. pisiformis), Entrophospora spp. (e.g., E. colombiana), Funneliformis spp. (e.g., F. mosseae), Gamarada spp. (e.g., G. debralockiae), Gigaspora spp. (e.g., G. gigantean, G. margarita), Glomus spp. (e.g., G. aggregatum, G. brasilianum, G. clarum, G. deserticola, G. etunicatum, G. fasciculatum G. intraradices, G. lamellosum, G. macrocarpum, G. monosporum, G. mosseae, G. versiforme), Gomphidius spp. (e.g., G. glutinosus), Hebeloma spp. (e.g., H. cylindrosporum), Hydnum spp. (e.g., H. repandum), Hymenoscyphus spp. (e.g., H. ericae), Inocybe spp. (e.g., I. bongardii, I. sindonia), Lactarius spp. (e.g., L. hygrophoroides), Lindtneria spp. (e.g., L. brevispora), Melanogaster spp. (e.g., M. ambiguous), Meliniomyces spp. (e.g., M. variabilis), Morchella spp., Mortierella spp. (e.g., M. polycephala), Oidiodendron spp. (e.g., O. maius), Paraglomus spp. (e.g., P. brasilianum), Paxillus spp. (e.g., P. involutus), Penicillium spp. (e.g., P. pinophilum, P. thomili), Peziza spp. (e.g., P. whitei), Pezoloma spp. (e.g., P. ericae); Phlebopus spp. (e.g., P. marginatus), Piloderma spp. (e.g., P. croceum), Pisolithus spp. (e.g., P. tinctorius), Pseudotomentella spp. (e.g., P. tristis), Rhizoctonia spp., Rhizodermea spp. (e.g., R. veluwensis), Rhizophagus spp. (e.g., R. irregularis), Rhizopogon spp. (e.g., R. luteorubescens, R. pseudoroseolus), Rhizoscyphus spp. (e.g., R. ericae), Russula spp. (e.g., R. livescens), Sclerocystis spp. (e.g., S. sinuosum), Scleroderma spp. (e.g., S. cepa, S. verrucosum), Scutellospora spp. (e.g., S. pellucida, S. heterogama), Sebacina spp. (e.g., S. sparassoidea), Setchelliogaster spp. (e.g., S. tenuipes), Suillus spp. (e.g., S. luteus), Thanatephorus spp. (e.g., T. cucumeris), Thelephora spp. (e.g., T. terrestris), Tomentella spp. (e.g., T. badia, T. cinereoumbrina, T. erinalis, T. galzinii), Tomentellopsis spp. (e.g., T. echinospora), Trechispora spp. (e.g., T. hymenocystis, T. stellulata, T. thelephora), Trichophaea spp. (e.g., T. abundans, T. woolhopeia), Tulasnella spp. (e.g., T. calospora), and Tylospora spp. (e.g., T. fibrillose).

In certain preferred embodiments, the subject invention utilizes endomycorrhizal fungi, including fungi from the phylum Glomeromycota and the genera Glomus, Gigaspora, Acaulospora, Sclerocystis, and Entrophospora. Examples of endomycorrhizal fungi include, but not are not limited to, Glomus aggregatum, Glomus brasilianum, Glomus clarum, Glomus deserticola, Glomus etunicatum, Glomus fasciculatum, Glomus intraradices (Rhizophagus irregularis), Glomus lamellosum, Glomus macrocarpum, Gigaspora margarita, Glomus monosporum, Glomus mosseae (Funneliformis mosseae), Glomus versiforme, Scutellospora heterogama, and Sclerocystis spp.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mucilaginosus, B. subtilis), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In specific embodiments, the one or more additional beneficial microorganisms are selected from, for example, nitrogen fixers (e.g., Azotobacter vinelandii), potassium mobilizers (e.g., Frateuria aurantia), and others including, for example, Myxococcus xanthus, Pseudomonas chlororaphis, Wickerhamomyces anomalus, Starmerella bombicola, Saccharomyces boulardii, Pichia occidentalis, Pichia kudriavzevii, Bacillus licheniform, Bacillus subtilis, and/or Meyerozyma guilliermondii.

Once applied to the soil, B. amy and/or combinations of B. amy with other microbial inoculants of the subject invention improve the mineralization of organic matter, increase nitrogen fixation needed for photosynthesis; increase phosphorous availability to crops while limiting its environmental leaching; improve salinity, pollutant content moisture retention, drainage, and nutrient dispersal of the rhizosphere; produce beneficial plant signaling metabolites; stimulate root mass by facilitating uptake of water and key nutrients; and/or boost plant biomass.

Advantageously, in some embodiments, the methods can also promote the formation of carbon sinks in soil by enhancing the sequestration of carbon in the soil, in above- and below-ground plant biomass, and in soil microbial biomass. Even further, in some embodiments, the methods can reduce the total greenhouse gas emissions produced during agricultural operations, for example, by reducing the amount of fertilizer and water required to produce crops.

In one embodiment, the inoculants can be customized by crop or geography to facilitate the robust colonization of beneficial microorganisms, which makes this technology ideal for proactively managing specific crops grown in vastly different soil ecosystems. The ability to customize microbial treatments to suit the needs of different soil ecosystems becomes even more important as a better understanding is developed of how complex microbial communities react to extreme temperatures, prolonged drought, variable rainfall, and other impacts stemming from climate change and intensive farming.

The mode of application according to the subject methods depends upon the formulation of the composition, and can include, for example, spraying, pouring, sprinkling, injecting, spreading, mixing, dunking, fogging and misting. Formulations can include, for example, liquids, dry and/or wettable powders, flowable powders, dusts, granules, pellets, emulsions, microcapsules, steaks, oils, gels, pastes and/or aerosols. In an exemplary embodiment, the composition is applied after the composition has been prepared by, for example, dissolving the composition in water.

In one embodiment, the site to which the composition is applied is the soil (or rhizosphere) in which plants will be planted or are growing (e.g., a crop, a field, an orchard, a grove, a pasture/prairie or a forest). The compositions of the subject invention can be pre-mixed with irrigation fluids, wherein the compositions percolate through the soil and can be delivered to, for example, the roots of plants to influence the root microbiome.

In one embodiment, the compositions are applied to soil surfaces, with or without water, where the beneficial effect of the soil application can be activated by rainfall, sprinkler, flood, or drip irrigation.

In one embodiment, the site is a plant or plant part. The composition can be applied directly thereto as a seed treatment, or to the surface of a plant or plant part (e.g., to the surface of the roots, tubers, stems, flowers, leaves, fruit, or flowers). In a specific embodiment, the composition is contacted with one or more roots of the plant. The composition can be applied directly to the roots, e.g., by spraying or dunking the roots, and/or indirectly, e.g., by administering the composition to the soil in which the plant grows (or the rhizosphere).

In one embodiment, wherein the method is used in a large scale setting, such as in a citrus grove, a pasture or prairie, a forest, a sod or turf farm, or an agricultural crop, the method can comprise administering the composition into a tank connected to an irrigation system used for supplying water, fertilizers, pesticides or other liquid compositions. Thus, the plant and/or soil surrounding the plant can be treated with the composition via, for example, soil injection, soil drenching, using a center pivot irrigation system, with a spray over the seed furrow, with micro-jets, with drench sprayers, with boom sprayers, with sprinklers and/or with drip irrigators. Advantageously, the method is suitable for treating hundreds of acres of land.

In one embodiment, wherein the method is used in a smaller scale setting, such as in a home garden or greenhouse, the method can comprise pouring the composition (mixed with water and other optional additives) into the tank of a handheld lawn and garden sprayer and spraying soil or another site with the composition. The composition can also be mixed into a standard handheld watering can and poured onto a site.

Plants and/or their environments can be treated at any point during the process of cultivating the plant. For example, the composition can be applied to the soil prior to, concurrently with, or after the time when seeds are planted therein. Seed application may be by, for example, a seed coating or by applying the composition to the soil contemporaneously with the planting of seeds. This may be automated by, for example, providing a device or an irrigation system that applies the microbe-based composition along with, and/or adjacent to, seeds at, or near, the time of planting the seeds. Thus, the microbe-based composition can be applied within, for example, 5, 4, 3, 2, or 1 day before or after the time of plantings or simultaneously with planting of the seeds. It can also be applied at any point thereafter during the development and growth of the plant, including when the plant is flowering, fruiting, and during and/or after abscission of leaves.

The subject methods can increase the above- and below-ground biomass of plants, including, for example, increased foliage volume, increased stem and/or trunk diameter, enhanced root growth and/or density, and/or increased numbers of plants. In one embodiment, this is achieved by improving the overall hospitability of the rhizosphere in which a plant's roots are growing, for example, by improving the nutrient and/or moisture retention properties of the rhizosphere.

Accordingly, the subject methods can benefit reforestation efforts, as well as efforts to restore depleted prairies and/or pastureland. In some embodiments, the amount of vegetation in a prairie/pastureland and/or forest has been depleted due to anthropogenic causes, such as over-grazing by livestock, logging, commercial, urban and/or residential development, and/or dumping. In some embodiments, the amount of vegetation is depleted due to fire, disease or other natural and/or environmental stressors.

Additionally, in one embodiment, the method can be used to inoculate soil and/or a plant's rhizosphere with a beneficial microorganism. The microorganisms of the subject microbe-based compositions can promote colonization of the roots and/or rhizosphere, as well as the vascular system of the plant, by, for example, beneficial bacteria, yeasts, and/or fungi.

In one embodiment, the promotion of colonization can lead to improved biodiversity of the soil microbiome. As used herein, improving the biodiversity refers to increasing the variety of microbial species within the soil.

For example, in one embodiment, the novel microbe strain of the subject composition, and others applied alongside, can colonize roots, the soil and/or the rhizosphere and encourage colonization of other nutrient-fixing microbes, such as Rhizobium and/or Mycorrhizae, and other endogenous and/or exogenous microbes that promote plant biomass accumulation.

In yet another embodiment, the method can be used to fight off and/or discourage colonization of the rhizosphere by soil microorganisms that are deleterious or that might compete with beneficial soil microorganisms. For example, in some embodiments, when more aerobic microorganisms are present in the soil, less anaerobic microorganisms, such as nitrate-reducing microorganisms, can thrive and produce deleterious atmospheric by-products, such as nitrous oxide.

In one embodiment, the method can be used for enhancing penetration of beneficial molecules through the outer layers of root cells, for example, at the root-soil interface of the rhizosphere.

The soil treatment compositions can be used either alone or in combination with other compounds for efficient enhancement of plant health, growth and/or yields, as well as other compounds for efficient treatment and prevention of plant pathogenic pests. For example, the methods can be used concurrently with sources of nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, and/or be applied with, for example, natural and/or chemical pesticides, repellants, herbicides, fertilizers, water treatments, non-ionic surfactants and/or soil amendments.

Preferably, the composition does not comprise and/or is not applied simultaneously with, or within 7 to 10 days before or after, application of the following compounds: benomyl, dodecyl dimethyl ammonium chloride, hydrogen dioxide/peroxyacetic acid, imazilil, propiconazole, tebuconazole, or triflumizole.

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g. microalga) and a plurality of plant cells that are largely differentiated into a colony (e.g. volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga edible by humans or used as a feed for animals or fish or marine animals, or consumed by humans, or used by humans (e.g., textile or cosmetics production), or viewed by humans (e.g., flowers or shrubs in landscaping or gardens) or any plant or alga, or a part thereof, used in industry or commerce or education.

Types of crop plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals). In certain specific embodiments, the crop plants include citrus, potatoes, corn, lettuce, hemp, turf, strawberries, tobacco, melons, and/or almonds.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant parts are understood as meaning all aerial and subterranean parts and organs of the plants such as shoot, leaf, flower and root, examples which may be mentioned being leaves, needles, stalks, stems, flowers, fruit bodies, fruits and seeds, but also roots, tubers and rhizomes. The plant parts also include crop material and vegetative and generative propagation material, for example cuttings, tubers, rhizomes, slips and seeds.

In some embodiments, the plant is a plant infected by a pathogenic disease or pest. In specific embodiments, the plant is infected with citrus greening disease and/or citrus canker disease, and/or a pest that carries such diseases.

### Use of the Subject Microbe Strain for Greenhouse Gas Reduction

In certain embodiments, B. amy can also be used to reduce deleterious atmospheric gases, such as carbon dioxide, methane, and nitrous oxide. In certain embodiments, the reduction in deleterious atmospheric gases is achieved via a reduction in methanogenic microbes of both animal and environmental origin.

In one embodiment B. amy and/or its growth by-products can disrupt methanogen biofilms. In one embodiment, the composition directly inhibits methanogens and/or the biological pathway involved in methanogenesis.

In one embodiment, a B. amy composition is applied to a lagoon. Manure lagoons are anaerobic basins filled with animal waste from livestock operations. Some lagoons are also used for pretreating industrial and/or municipal wastewaters. Due to the presence of methanogenic microorganisms that feed on the organic matter in the wastewater, lagoons are a large source of methane emissions.

In one embodiment, a B. amy composition is applied to a rice paddy. Standard rice growing practice entails flooding of rice fields during the growing season. During flooding, however, methanogenic microorganisms thrive on decaying organic matter in the water, thus releasing methane emissions in large amounts.

By applying a composition of the subject invention to the water and other liquids in a lagoon or a rice paddy, the subject methods can effectively reduce atmospheric methane emissions through, for example, the control of methanogenic microorganisms.

In certain embodiments, a B. amy composition can be applied to the digestive system of livestock or another animal, including domesticated pets. The composition can be applied as, for example, a spore-form probiotic, to enhance body weight gain, to promote feed intake and conversion, and to increase growth hormone levels.

Additionally, when administered to the digestive system of a livestock animal, B. amy can also be used for reducing the production of enteric greenhouse gases (e.g., methane and carbon dioxide) and/or greenhouse gas precursors (e.g., organic nitrogen). B. amy can promote the growth of other beneficial microbes (e.g., fatty acid producers, which can inhibit methanogens) while decreasing the amount of potential pathogenic and/or methanogenic microbes in an animal's gut.

In some embodiments, B. amy can be applied to the digestive system of livestock or another animal in combination with one or more other microbes, including, for example, Pleurotus ostreatus, Lentinula edodes, Trichoderma viridae, Wickerhamomyces anomalus, Saccharomyces cerevisiae, Saccharomyces boulardii, Starmerella bombicola, Meyerozyma guilliermondii, Pichia occidentalis, Monascus purpureus, Acremonium chrysogenum, Myxococcus xanthus, Bacillus subtilis and/or Bacillus licheniformis.

In some embodiments, B. amy can be applied to the digestive system of livestock or another animal in combination with a prebiotic such as, for example, dry animal fodder, straw, hay, alfalfa, grains, forage, grass, fruits, vegetables, oats, or crop residue.

In some embodiments, B. amy can be applied to the digestive system of livestock or another animal in combination with a saturated long chain fatty acid such as, for example, stearic acid, palmitic acid and/or myristic acid.

In some embodiments, B. amy can be applied to the digestive system of livestock or another animal in combination with a germination enhancer such as, for example, L-alanine, L-leucine or manganese. This is particularly useful in the case the B. amy is applied in spore-form.

In some embodiments, the composition can comprise additional components known to reduce methane in the livestock animal's digestive system, such as, for example, seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

In one embodiment, the subject composition can comprise one or more additional substances and/or nutrients to supplement the livestock animal's nutritional needs and promote health and/or well-being in the livestock animal, such as, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

The composition can be administered enterally and/or parenterally to the animal's digestive system. For example, the composition can be administered to the animal orally, via the animal's feed, a salt lick/mineral block, and/or drinking water; via endoscopy; via direct injection into one or more parts of the digestive system; via suppository; via fecal transplant; and/or via enema.

“Domesticated” animals are species that have been influenced, bred, tamed, and/or controlled over a sustained number of generations by humans, such that a mutualistic relationship exists between the animal and the human. Domesticated animals can be “pets,” which include animals that are raised and cared for by a human for protection and/or companionship, such as, for example, dogs, cats, horses, pigs, primates, birds, rodents and other small mammals, reptiles and fish. “Livestock” animals, are domesticated animals raised in an agricultural or industrial setting to produce commodities such as food, fiber and labor. Types of animals included in the term livestock can include, but are not limited to, alpacas, llamas, pigs (swine), horses, mules, asses, camels, dogs, ruminants, chickens, turkeys, ducks, geese, guinea fowl, and squabs.

In certain embodiments, the livestock animals are “ruminants,” or mammals that utilize a compartmentalized stomach suited for fermenting plant-based foods prior to digestion with the help of a specialized gut microbiome. Ruminants include, for example, bovines, sheep, goats, ibex, giraffes, deer, elk, moose, caribou, reindeer, antelope, gazelle, impala, wildebeest, and some kangaroos.

In specific exemplary embodiments, the livestock animals are bovine animals, which are ruminant animals belonging to the subfamily Bovinae, of the family Bovidae. Bovine animals can include domesticated and/or wild species. Specific examples include, but are not limited to, water buffalo, anoa, tamaraw, auroch, banteng, guar, gayal, yak, kouprey, domestic meat and dairy cattle (e.g., Bos taurus, Bos indicus), ox, bullock, zebu, saola, bison, buffalo, wisent, bongo, kudu, kewwel, imbabala, kudu, nyala, sitatunga, and eland.

Advantageously, in preferred embodiments, the methods result in a direct inhibition of methanogenic bacteria and/or symbionts thereof, disruption of methanogenic biofilms, and/or disruption of the biological pathway involved in methanogenesis in the livestock animal's digestion system, for example, the rumen, stomach and/or intestines.

In some embodiments, the methods can be utilized for enhancing the overall health of a livestock animal, for example, by contributing to a healthy gut microbiome, improving digestion, increasing feed-to-muscle conversion ratio, increasing milk production and quality, reducing and/or treating dehydration and heat stress, modulating the immune system, and increasing life expectancy.

In certain embodiments, the methods also reduce GHG emissions from the livestock animal's waste (e.g., urine and/or manure). In some embodiments, B. amy can survive transport through the digestive system and is excreted with the animal's waste, where it continues inhibiting methanogens and/or symbionts thereof, disrupting methanogenic biofilms, disrupting the biological pathways involved in methanogenesis, and/or compensating for H₂ acceptor loss. The composition can be administered to the livestock animal's digestive system and/or directly to the waste product.

In certain specific embodiments, the composition can be administered directly to a manure lagoon, waste pond, tailing pond, tank or other storage facility where livestock manure is stored and/or treated. Advantageously, in some embodiments, B. amy and/or combinations thereof with other microorganisms can facilitate an increased decomposition rate for the manure while reducing the amount of methane and/or nitrous oxide emitted therefrom. Furthermore, in some embodiments, applying the composition to the manure enhances the value of the manure as an organic fertilizer due to the ability of the microorganisms to inoculate the soil to which the manure is applied. The microbes then grow and, for example, improve soil biodiversity, enhance rhizosphere properties, and enhance plant growth and health.

In some embodiments, the methods of the subject invention can be utilized by a farmer and/or livestock producer for reducing carbon credit usage. Thus, in certain embodiments, the subject methods can further comprise conducting measurements to assess the effect of the method on reducing the generation of methane, carbon dioxide and/or other deleterious atmospheric gases, and/or precursors thereof (e.g., nitrogen and/or ammonia), and/or to assess the effect of the method on the emissions of soil-borne GHG and on the control of methanogens and/or protozoa in the livestock animal's digestive system and/or waste, using standard techniques in the art.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where a resulting microbe-based product will be used (e.g., a free-range cattle pasture). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a livestock production facility), preferably within 300 miles, more preferably within 200 miles, even more preferably within 100 miles. Advantageously, this allows for the compositions to be tailored for use at a specified location. The formula and potency of microbe-based compositions can be customized for specific local conditions at the time of application, such as, for example, which animal species is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Transformed Microbes

In one embodiment, the subject invention pertains to the genetic transformation of host cells (e.g., Gram positive or Gram negative bacteria) so as to provide these bacteria with the ability to produce a lipopeptide mixture comprising surfactin, lichenysin, fengycin, and iturin A. Thus, in some embodiments, the subject invention allows the use of recombinant strains of Gram positive and/or Gram negative bacteria for the production of a lipopeptide.

In one aspect of the subject invention yeast, Gram negative and/or Gram positive organisms are transformed with one or more nucleic acid sequences encoding for one or more biological mechanisms capable of synthesizing the lipopeptide mixture. The organisms that are transformed may, or may not, contain a naturally occurring nucleic acid sequence of this type.

The host cell may be, selected from, for example, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas fluorescens, Pseudomonas ovalis, Pseudomonas stutzeri, Pseudomonas acidovolans, Pseudomonas mucidolens, Pseudomonas testosterone, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Thermotoga martima, Geobacillus sterothermophilus and so forth (in certain embodiments, thermotolerant microorganisms, such as a thermotolerant B. coagulans strain are preferred).

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Co-Cultivation for Enhanced Lipopeptide Production

In one embodiment, compositions comprising lipopeptides (e.g., surfactin, iturin and/or fengycin) are produced using co-cultivation of B. amy and Myxococcus xanthus. When grown together, the species try to inhibit one another, thereby producing high concentrations of lipopeptides.

B. amy inoculum is grown in a small-scale reactor for 24 to 48 hours. Myxococcus xanthus inoculum is grown in a 2L working volume seed culture flask for 48 to 120 hours. A fermentation reactor is inoculated with the two inocula. The nutrient medium comprises:

| | |
|---|---|
| Glucose | 1 g/L to 5 g/L |
| Casein peptone | 1 g/L to 10 g/L |
| K₂HPO₄ | 0.01 g/L to 1.0 g/L |
| KH₂PO₄ | 0.01 g/L to 1.0 g/L |
| MgSO₄•7H₂O | 0.01 g/L to 1.0 g/L |
| NaCl | 0.01 g/L to 1.0 g/L |
| CaCO₃ | 0.5 g/L to 5 g/L |
| Ca(NO₃)₂ | 0.01 g/L to 1.0 g/L |
| Yeast extract | 0.01 g/L to 5 g/L |
| MnCl₂•4H₂O | 0.001 g/L to 0.5 g/L |
| Teknova trace element | 0.5 ml/L to 5 ml/L |

Fine grain particulate anchoring carrier is suspended in the nutrient medium. The carrier comprises cellulose (1.0 to 5.0 g/L) and/or corn flour (1.0 to 8.0 g/L).

The B. amy produces lipopeptides into the liquid fermentation medium. The entire culture can be used as-is, or the culture can be processed and, optionally, the lipopeptides purified.

### Example 2—Disinfecting Cleaning Composition

The lipopeptide mixture produced by B. amy can be used in environmentally-friendly cleaning compositions and to enhance antimicrobial activity of other biosurfactants. Cleaning compositions were tested for their ability to control Gram-negative E. coli. Reduction in optical density at 600 nm (OD) was measured for cultures treated with each of the following compositions:

TABLE 1

| | | OD₆₀₀ % reduction |
|---|---|---|
| # | Samples tested against E. coli | after 2 hours |
| 1 | Control (culture with no biosurfactant) | |
| 2 | 300 ppm lipopeptide mixture | 0 |
| 3 | 250 ppm lactonic SLP (natural) | 3.4 |
| 4 | 250 ppm linear SLP isopropyl ester | 86.4 |
| 5 | 250 ppm linear SLP ethyl ester | 89.8 |
| 6 | 250 ppm linear SLP butyl ester | 91.5 |
| 7 | 50 ppm silver-SLP nanoparticles | 99.0 |
| 8 | 300 ppm lipopeptide mixture + | 99.8 |
| | 50 ppm silver-SLP nanoparticles | |
| 9 | 100 ppm linear butyl ester + | 100 |
| | 100 ppm lactonic SLP (natural) | |

Table 1 shows the amount of OD reduction from least to greatest, where sample 1 performed the worst and sample 9 performed the best. The lipopeptide mixture of sample 2 (comprising surfactin, lichenysin, fengycin, and iturin A) was essentially ineffective on its own, but when combined with 50 ppm silver-SLP nanoparticles (sample 8), the effect of silver-SLP nanoparticles was enhanced versus on their own (sample 7).

Cleaning compositions according to embodiments of the subject invention were also tested for their ability to control a Gram-positive Staphylococcus sp. Reduction in optical density at 600 nm (OD) was measured for cultures treated with each of the following compositions:

Table 2 shows the amount of OD reduction from least to greatest, where sample 1 performed the worst and samples 8 and 9 performed the best.

TABLE 2

| | Samples tested against | OD₆₀₀ % reduction |
|---|---|---|
| # | Staphylococcus sp. | after 2 hours |
| 1 | Control (culture with no biosurfactant) | |
| 2 | 250 ppm linear SLP butyl ester | 88.1 |
| 3 | 150 ppm lipopeptide mixture | 92.0 |
| 4 | 50 ppm linear SLP isopropyl ester | 91.5 |
| 5 | 5 ppm lactonic SLP | 93.2 |
| 6 | 100 ppm linear SLP ethyl ester | 98.3 |
| 7 | 5 ppm silver-SLP nanoparticles | 99.9 |
| 8 | 100 ppm linear butyl ester + | 100 |
| | 100 ppm lactonic SLP | |
| 9 | 100 ppm lactonic butyl ester + | 100 |
| | 100 ppm lactonic SLP | |

1-15. (canceled)

16. A method for reducing a deleterious atmospheric gas, which comprises applying a composition comprising a Bacillus amyloliquefaciens var. locus (“B. amy”) of having accession number NRRL B-67928 to a source of the deleterious atmospheric gas.

17. The method of claim 16, wherein the deleterious atmospheric gas is methane, carbon dioxide, and/or nitrous oxide.

18. The method of claim 17, wherein the source of methane is a lagoon or rice paddy having methanogenic microbes therein, and wherein the methanogenic microbes are controlled.

19. The method of claim 17, wherein the source of methane, carbon dioxide and/or nitrous oxide is a livestock or other animal's digestive system.

20. The method of claim 19, further comprising applying one or more of the following components to the livestock or other animal's digestive system: long chain saturated fatty acids; germination enhancers; valine; HMG-CoA reductase inhibitors; seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

21. The method of claim 19, wherein a methanogenic microorganism and/or a methanogen biofilm in the livestock or other animal's digestive system is controlled.

22. The method of claim 17, wherein the source of nitrous oxide is soil containing nitrogen-based fertilizers, and wherein the composition improves the bioavailability of the nitrogen-based fertilizer to plants, as well as reduces the amount of fertilizer required in future applications, thereby reducing the amount of residual nitrous oxide precursors present in soil in the form of excess fertilizers.

23. A method for enhancing the sequestration of carbon, which comprises applying a composition comprising B. amyloliquefaciens to soil in which a plant is or will be planted, wherein above- and below-ground biomass of the plant is enhanced, soil microbial biomass is enhanced, and total organic carbon content of the soil is enhanced, thereby creating a carbon sink.

24. The method, according to claim 23, wherein said B. amyloliquefaciens has accession number NRRL B-67928.
